(19) United States
(12) Patent Application Publication
(10) Pub. No.: US 2026/0174496 A1
(43) Pub. Date: Jun. 25, 2026

(54) SYSTEM AND METHOD FOR DETERMINING STATE OF RF DEVICES USING IMPEDANCE MEASUREMENTS

(71) Applicant: Boston Scientific Scimed, Inc., Maple Grove, MN (US)

(72) Inventors: Steven Kinio, Pickering (CA); Christian Balkovec, Cambridge (CA); Laurentiu Murtescu, Vaughan (CA); Deep Patel, Waterdown (CA)

(21) Appl. No.: 19/540,301

(22) Filed: Feb. 13, 2026

Related U.S. Application Data
(63) Continuation of application No. 18/912,056, filed on Oct. 10, 2024.
(60) Provisional application No. 63/592,008, filed on Oct. 20, 2023.

(51) Int. Cl. A61B 18/14; A61B 18/00; A61B 18/16

(52) U.S. Cl. CPC A61B 18/1487; A61B 18/16; A61B 2018/00684; A61B 2018/00845; A61B 2018/00869; A61B 2018/00875; A61B 2018/00988

(57) ABSTRACT

A radiofrequency (RF) generator for use in a tissue puncture system for puncturing a tissue in a body is disclosed. The RF generator includes an RF energy source to couple to an active electrode of an RF puncture device and to couple to a return electrode and a controller coupled to the RF energy source. The controller causes the RF energy source to generate each of a puncture signal and a test signal, the test signal having a lower power than the puncture signal, measures an impedance of the test signal, determine a state of the active electrode based on the impedance measurement, and displays the state on a display device.

### CROSS REFERENCE TO RELATED APPLICATIONS

This application is a continuation of U.S. patent application Ser. No. 18/912,056 entitled “SYSTEM AND METHOD FOR DETERMINING STATE OF RF DEVICES USING IMPEDANCE MEASUREMENTS,” filed Oct. 10, 2024, which claims priority to U.S. Provisional Patent Application No. 63/592,008 entitled “SYSTEM AND METHOD FOR DETERMINING STATE OF RF DEVICES USING IMPEDANCE MEASUREMENTS,” filed Oct. 20, 2023, which is hereby incorporated by reference in its entirety.

### TECHNICAL FIELD

The present disclosure relates to medical devices and systems for use in percutaneous or interventional procedures including surgery such as electrophysiology procedures. More specifically, this disclosure relates to electrosurgical units, such as radiofrequency (RF) generators, electrosurgical systems, and methods, that provide for impedance measurements.

### BACKGROUND

Catheters are often used to provide general access into a patient's body using minimally invasive techniques. In some examples, a catheter can be used to create a channel through a region of the body. For instance, punctures in tissues can provide access for medical tools used in various medical interventions. In one example, a pericardium layer of a patient can be punctured to provide for epicardial access, such as to create an access point to insert tools for epicardial ablation. In another example, electrosurgical devices are applied to remove accumulation of atheromatous material on the inner walls of vascular lumens, which results in atherosclerosis. In one technique, an electrosurgical device is applied to puncture through the vascular occlusion without affecting the vessel walls. Another example is a transseptal puncture in a cardiac procedure. The left atrium is a difficult cardiac chamber to reach percutaneously. Although the left atrium can be reached via the left ventricle and mitral valve, the catheter is manipulated through two U-turns, which can be cumbersome. the transseptal puncture is a technique of creating a small surgical passage through the atrial septum, or wall in the heart between the left and right atrium, through which a catheter can be fed. The atrial septum is punctured and dilated via tools. The transseptal puncture permits a direct route to the left atrium via the intra-atrial septum and systematic venous system. Increasing larger and complex medical devices can be passed into the right atrium.

Punctures, such as transseptal punctures, can be performed with the aid of guidewires having electrodes energized with a suitable power source such as an electrically coupled power generator to provide the source of RF energy in a manner like other electrosurgical devices. Typical electrosurgical devices apply an electrical potential difference or a voltage difference between an active electrode and a return electrode on a patient's grounded body in a monopolar arrangement or between an active electrode and a return electrode on the device in bipolar arrangement to deliver the RF energy to the area where tissue is to be affected. Electrosurgical devices pass RF energy through tissue between the electrodes to puncture tissue with plasma formed on the energized electrode. Tissue that contacts the plasma experiences a rapid vaporization of cellular fluid to produce a cutting effect. Electrical energy can be applied to the electrodes either as a train of high frequency pulses or as a continuous signal typically in the radiofrequency (RF) range to perform the cutting or puncturing techniques.

### SUMMARY

In Example 1, a radiofrequency generator for use in a tissue puncture system for puncturing a tissue in a body, the radiofrequency generator comprising: a radiofrequency energy source configured to couple to an active electrode of an radiofrequency puncture device and configured to couple to a return electrode; and a controller coupled to the radiofrequency energy source, the controller configured to: cause the radiofrequency energy source to generate each of a puncture signal and a test signal, the test signal having a lower power than the puncture signal; measure an impedance of the test signal; determine a state of the active electrode based on the impedance measurement; display the state on a display device.

In Example 2, the radiofrequency generator of Example 1, wherein the impedance includes complex impedance having magnitude and phase angle information.

In Example 3, the radiofrequency generator of any of Examples 1 and 2, wherein the state of the active electrode is based on the radiofrequency puncture device.

In Example 4, the radiofrequency generator of any of Examples 1-3, wherein the electrosurgical device includes the active electrode disposed in a dilator/sheath assembly and the state of the active electrode is based on a position of the active electrode with respect to the dilator/sheath assembly.

In Example 5, the radiofrequency generator of Example 4, the state of the active electrode is based on a position of the active electrode with respect to the tissue.

In Example 6, the radiofrequency generator of any of Examples 1-5, wherein the test signal includes a frequency above 100 kHz.

In Example 7, the radiofrequency generator of any of Examples 1-6, wherein the test signal includes a first frequency, and the puncture signal includes a second frequency.

In Example 8, the radiofrequency generator of Example 7, wherein the first frequency is the same as the second frequency.

In Example 9, the radiofrequency generator of any of Examples 1-8, wherein the state of the active electrode based on the impedance measurement is selected from one of open circuit, active electrode positioned proximally in dilator/sheath assembly, active electrode positioned distally in dilator/sheath assembly, active electrode at dilator tip of dilator/sheath assembly, active electrode extended from dilator tip of dilator/sheath assembly and in contact with target tissue, and active electrode extended from dilator tip of dilator/sheath assembly and in contact with fluid.

In Example 10, the radiofrequency generator of any of Examples 1-9, wherein the state of the active electrode is determined from a lookup table.

In Example 11, the radiofrequency generator of Example 10, wherein the lookup table is a multidimensional lookup table having impedance magnitude and phase angle as inputs.

In Example 12, radio frequency generator of any of Examples 10-11, wherein the lookup table includes test signal frequency as an input.

In Example 13, the radio frequency generator of any of Examples 1-12, wherein the controller is configured to load data to determine the state into a memory device of the controller.

In Example 14, the radio frequency generator of Example 13, wherein the data is stored on a memory device coupled to the radiofrequency puncture device.

In Example 15, the radiofrequency device of any of Examples 1-14, wherein the radio frequency device is included in the puncture system having the RF puncture device.

In Example 16, a radiofrequency generator for use in a tissue puncture system for puncturing a tissue in a body, the radiofrequency comprising: a radiofrequency (RF) circuit configured to couple to an active electrode of an RF puncture device and configured to couple to a return electrode, the RF circuit configured to generate each of a puncture signal and a test signal, the test signal having a lower power than the puncture signal; and a controller coupled to the RF circuit, the controller configured to: measure an impedance of the test signal; determine a state of the active electrode based on the impedance measurement; and display the state on a display device.

In Example 17, the radiofrequency generator of Example 16, wherein the impedance includes complex impedance having magnitude and phase angle information.

In Example 18, the radiofrequency generator of Example 16, wherein the test signal includes a frequency above 100 kHz.

In Example 19, the radiofrequency generator of Example 16, wherein the test signal includes a first frequency, and the puncture signal includes a second frequency.

In Example 20, the radiofrequency generator of Example 19, wherein the first frequency is the same as the second frequency.

In Example 21, the radiofrequency generator of Example 16, wherein the state of the active electrode is based on data loaded into a memory device of the controller.

In Example 22, the radio frequency generator of Example 21, wherein the data is stored on a non-transitory memory device coupled to the RF puncture device.

In Example 23, the radiofrequency generator of Example 16, wherein the state of the active electrode is determined from a lookup table.

In Example 24, the radiofrequency generator of Example 23, wherein the lookup table is a multidimensional lookup table having impedance magnitude and phase angle as inputs.

In Example 25, the radio frequency generator of Example 23, wherein the lookup table includes test signal frequency as an input.

In Example 26, the radiofrequency generator of Example 16, wherein the state of the active electrode based on the impedance measurement is selected from one of open circuit, active electrode positioned proximally in dilator/sheath assembly, active electrode positioned distally in dilator/sheath assembly, active electrode at dilator tip of dilator/sheath assembly, active electrode extended from dilator tip of dilator/sheath assembly and in contact with target tissue, and active electrode extended from dilator tip of dilator/sheath assembly and in contact with fluid.

In Example 27, a tissue puncture system for puncturing a tissue in body, the tissue puncture system comprising: a puncture device having an active electrode; and a radiofrequency (RF) generator coupled to the puncture device, the RF generator comprising: an RF circuit configured to couple to the active electrode of the puncture device and configured to couple to a return electrode, the RF circuit configured to generate each of a puncture signal and a test signal, the test signal having a lower power than the puncture signal; and a controller coupled to the RF circuit, the controller configured to measure an impedance of the test signal; determine a state of the active electrode based on the impedance measurement; and display the state on a display device.

In Example 28, the tissue puncture system of Example 27, wherein the puncture device is an RF puncture device.

In Example 29, the tissue puncture system of Example 28, wherein the return electrode includes a patch electrode, and the tissue puncture system is configured to operate in a monopolar mode.

In Example 30, the tissue puncture system of Example 28, wherein the RF puncture device includes a transseptal guidewire disposed within a dilator/sheath assembly.

In Example 31, the tissue puncture system of Example 27, wherein the state of the active electrode is determined from a lookup table.

In Example 32, the tissue puncture system of Example 31, wherein the lookup table is stored on a non-transitory memory device coupled to the puncture device.

In Example 33, a method for use in a tissue puncture system, the tissue puncture system including a radiofrequency energy source coupled to an active electrode of a puncture device and to a return electrode, the method comprising: causing the radiofrequency (RF) energy source to generate each of a puncture signal and a test signal, the test signal having a lower power than the puncture signal; measuring an impedance of the test signal; determining a state of the active electrode based on the impedance measurement; and displaying the state on a display device.

In Example 34, the method of Example 33, wherein the measuring the impedance of the test signal includes measure a complex impedance having magnitude and phase angle information.

In Example 35, the method of Example 33, wherein the determining the state of the active electrode includes selecting the state of the active electrode from one of open circuit, active electrode positioned proximally in dilator/sheath assembly, active electrode positioned distally in dilator/sheath assembly, active electrode at dilator tip of dilator/sheath assembly, active electrode extended from dilator tip of dilator/sheath assembly and in contact with target tissue, and active electrode extended from dilator tip of dilator/sheath assembly and in contact with fluid.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary electrosurgical system for performing an electrosurgical puncture, such as a transseptal puncture.

FIG. 2 is a graph illustrating impedance of an electrical load as measured by a generator during a transseptal puncture workflow.

FIG. 3A is a table illustrating impedance magnitudes for various states of a transseptal guidewire at various frequencies.

FIG. 3B is a table illustrating impedance phase angles for various states of a transseptal guidewire at various frequencies.

FIG. 4 is a block diagram illustrating an electrosurgical generator configured for use with the electrosurgical system of FIG. 1.

FIG. 5 is a block diagram illustrating a process of the electrosurgical generator of FIG. 4.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

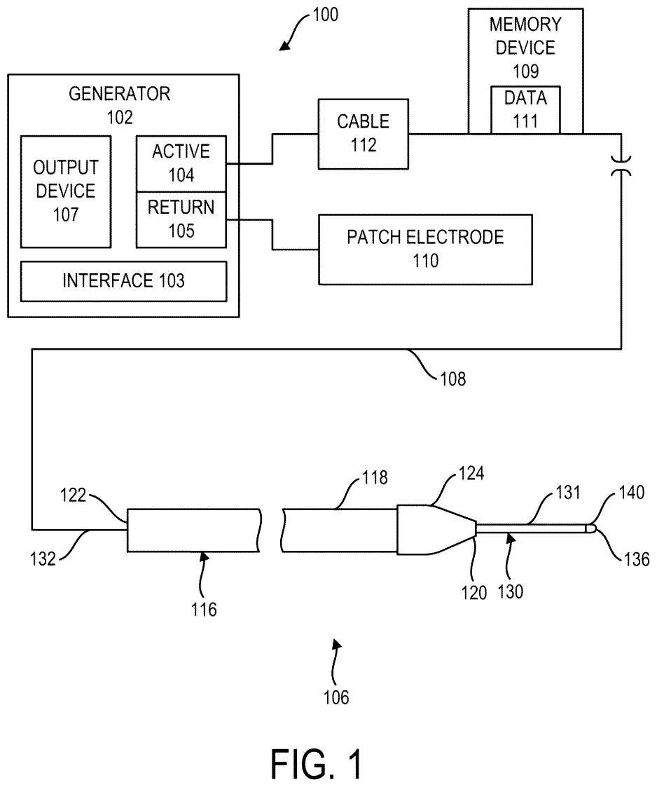

FIG. 1 illustrates an embodiment of an electrosurgical system 100 to facilitate vascular access to a heart and provide catheter positioning within cardiac anatomy. The embodiment of the medical system 100 includes an electrosurgical unit, such as an electrosurgical generator 102 and an electrosurgical device, such as an electrosurgical puncture device 106. In one example, the electrosurgical puncture device 106 includes an electrosurgical transseptal guidewire 108. In the illustration, the electrosurgical transseptal guidewire 108 is electrically coupled to the electrosurgical generator 102 via cable 112, such as to an active connector 104 on the electrosurgical generator 102. The electrosurgical generator 102 is configured to provide a source of energy, such as radiofrequency (RF) energy to the electrosurgical transseptal guidewire 108 via the cable 112. In some embodiments, the system 100 includes a ground pad electrode, or indifferent (dispersive) patch electrode 110 electrically coupled to the electrosurgical generator 102, such as to a return connector 105 on the electrosurgical generator 102, for use with the electrosurgical transseptal guidewire 108 in a monopolar configuration. In some embodiments, the electrosurgical transseptal guidewire is implemented in a bipolar configuration using a pair of electrodes on the guidewire and without a separate patch electrode.

The electrosurgical generator 102 is configured to provide the source of RF energy to the electrosurgical transseptal guidewire 108 for a puncture operation with the electrosurgical device 106. The electrosurgical generator 102 includes an interface 103 including a set of user accessible controls, device connectors such as active connector 104 and a return connector 105, and an output device 107 such as a display device, speakers, and lights. During a monopolar puncture operation of electrosurgical generator 102, a first electrode, often referred to as the active electrode, is provided with the electrosurgical device 106 in general and with the transseptal guidewire 108 in the illustration while a second electrode, such as patch electrode 110, is typically located on the back, buttocks, upper leg, or other suitable anatomical location of the patient during surgery. In such a configuration, the patch electrode 110 is often referred to as a patient return electrode. An electrical circuit of RF energy is formed between the active electrode and the patch electrode 110 through the patient, which is used to puncture tissue at the active electrode. For example, RF energy for a puncture function in a monopolar mode may be provided at a relatively low voltage and a continuous current (100% on, or 100% duty cycle). At a power setting of 50 Watts for puncturing (although instantaneous power may be higher), voltage can range from approximately 164 to 400 volts root mean square (RMS). The electrosurgical generator 102 can include a plurality of functions and provide a programmed and custom settings via an interface and be couplable to a suite of electrosurgical devices in addition to the transeptal guidewire 108. In one example, the electrosurgical generator 102 provides RF energy to the active electrode as an alternating current having a frequency in the range of 100 kHz to 10 MHz. Typically, this energy is applied in the form of a continuous sinusoidal puncture signal. In some embodiments, the energy is applied in bursts of pulses. The individual pulses in each burst of a pulsed puncture signal typically each have a duration of 300 milliseconds with an interval between pulses of 700 milliseconds but can vary such as based on parameters of the connected electrosurgical device 106. The actual pulses are often sinusoidal or square waves and bi-phasic, that is alternating positive and negative amplitudes.

In one example, the electrosurgical generator 102 provides the power to the electrosurgical puncture device 106, but the actual power level delivered to the electrosurgical puncture device 106 can be selected via controls on the electrosurgical puncture device 106 rather than controls on the electrosurgical generator 102. In another example, the electrosurgical generator 102 can be programmed to provide power levels within a selected range of power, and the electrosurgical puncture device 106 is used to select an output power level within the preprogrammed range. For instance, the electrosurgical generator 102 can be programmed to provide monopolar energy for a puncture function in a first range of power settings as well as voltage-based controls to target a specific voltage. The electrosurgical generator 102 can be programmed to provide monopolar energy for another function in a second range of power or voltage settings, which second range may be the same as, different than, or overlap the first range. In some embodiments, the user may then select the function and adjust the power or voltage setting within the range using controls on the electrosurgical puncture device 106 rather than using controls on the electrosurgical generator 102.

In one embodiment, the electrosurgical generator 102 can program and select particular controls, or ranges of controls, based on the particular configuration of the electrosurgical transeptal guidewire 108. The transseptal guidewire of the embodiment includes a memory device 109 (non-transitory memory) storing a set of parameters 111 associated with the transseptal guidewire 108. The electrosurgical generator 102 is configured to read the parameters to program the controls to be suited for the associated transseptal guidewire. The memory device 109 can store the parameters 111 in various memory segments having lookup tables or other data structures to provide data to be loaded into a memory device in the electrosurgical generator 102 and read by a controller of the electrosurgical generator to affect operation. Example parameters 111 can include model number of the transseptal guidewire 108, acceptable power levels signals applied to the transseptal device 108, whether the transseptal device is configured for single use or multiple uses, as well as other parameters. In some embodiments, the electrosurgical generator 102 can be programmed to write to memory segments on the memory device 109 as well as read the memory device 109.

The illustrated electrosurgical puncture device 106 includes the electrosurgical transseptal guidewire 108 and a delivery component 116. While embodiments of the disclosure are described with reference to punctures in tissue with a transseptal guidewire for illustration, the features of the disclosure can be used with other electrosurgical devices including other transseptal surgical devices such as needle-based platforms. The delivery component 116 includes an elongated shaft 118 having a shaft distal tip 120. The elongated shaft 118 defines a longitudinally extending axial lumen 122. The electrosurgical transseptal guidewire 108 is adapted to be disposed within the lumen 122 and coupled to the RF energy source. In some embodiments, the delivery component 116 can include an elongate sheath, and the electrosurgical transseptal guidewire 108 is disposed within the sheath. In another embodiment, the delivery component 116 can include a dilator/sheath assembly, and the electrosurgical transseptal guidewire 108 is disposed within the dilator/sheath assembly. For instance, the elongated shaft 118 includes a distal tapered portion 124 with an enlargement of cross-sectional area with respect to the shaft distal tip 120. As the distal tapered portion 124 is passed through an aperture from the shaft distal tip 120, the enlargement of cross-sectional area dilates the aperture. The dilator can be configured as a straight dilator, as illustrated, or a curved dilator. The elongated shaft 118 can be made from various materials including insulative materials such as high-density polyethylene (HDPE). The shaft 118 and distal tip can include various materials such as metal hypotubes as well.

The electrosurgical transseptal guidewire 108 includes a puncture wire shaft 130 with a puncture wire proximal portion 132 and a puncture wire distal portion 134 having a puncture wire distal tip 136. The puncture wire distal tip 136 includes a puncture electrode 140 adapted to deliver the RF energy. The puncture electrode 140 is configured as the active electrode. The puncture wire proximal portion 132 includes an end connector 142 configured to electrically couple to cable 112 and receive an RF signal from the electrosurgical generator 102. In one example, the electrosurgical transeptal guidewire 108 can be coupled to and uncoupled from the cable 112 depending on whether the electrosurgical transeptal guidewire 108 is used as an electrosurgical puncture device or as an exchange rail, for instance. The transseptal guidewire 108 is configured to conduct the RF signal from the proximal portion 132 along the puncture wire shaft 130 to the electrode 140. In some embodiments, the puncture wire shaft 130 is constructed from an electrically conductive material having an insulative outer coating. In some embodiments, the electrically conductive material is a flexible, shape memory material such as a nickel titanium alloy or nitinol. The exposed electrode 140 is configured to apply the RF energy, such as to puncture tissue.

In the illustrated example, the electrosurgical transseptal guidewire 108 is configured as a multifunction conductive guidewire. For instance, the transseptal guidewire 108 can be used, without exchanges, as a guidewire, a transseptal puncture device, and as an exchange rail for delivering therapy sheaths. Such embodiments provide efficiencies to medical procedures as the transseptal guidewire 108 performs multiple functions and reduces the amount of device exchanges in the medical procedure. The transseptal guidewire 108 includes a distal tip 136 extendable from the delivery component distal end 120 such that the delivery component 116 is retractable from the patient over the guidewire 108 with the guidewire distal tip 136 disposed within the heart. The transseptal guidewire 108 is sufficiently thin and flexible to access the various chambers of the heart. The electrode 140 on the puncture wire distal tip 136 is operable to deliver RF energy to puncture the atrial septum from the right atrium, and the distal portion 134 of the puncture wire shaft 130 can be advanced through the puncture. Once advanced through the puncture and sufficiently extended from within the delivery component 136, the distal portion 134 is biased to form a coil for anchoring the transseptal guidewire 108 beyond the puncture. The delivery component 116 is retractable from the patient over the transseptal guidewire 108 with the distal tip 136 still disposed within the heart. The transseptal guidewire 108 can also support the installation of therapy devices to a therapy location in the patient's heart, such as tubular members or other catheters and for advancing other devices within the heart.

In an anticipated use of the system 100, the electrosurgical device 106 is coupled to the RF generator 102, and if the electrosurgical device 106 is to be configured in a monopolar mode, the patch electrode 110 is coupled to the patient. The RF generator 102 can be set to a puncture mode, such as an energy output of approximately 10 watts. In some examples, femoral access is obtained via a conventional percutaneous needle, and the transseptal guidewire 108 is inserted into the vasculature and advanced to the superior vena cava. The shaft distal tip 120 of the delivery component 116 is advanced over the proximal portion 132 of the guidewire 108, and the distal tapered portion 124 of the delivery component shaft 118 is advanced over the guidewire 108 to the superior vena cava. Under visualization, the distal tapered portion 124 is moved from the superior vena cava to the right atrial septum and then to the fossa ovalis of the heart. Once the delivery component distal tip 120 is confirmed at the fossa ovalis, the electrode 140 of the transseptal guidewire 108 is advanced from the delivery component distal tip 120. In one example, the exposed puncture electrode 140 of the transseptal guidewire 108 is extended a few millimeters from the delivery component distal tip 120 to tent the heart tissue, and the transseptal guidewire 108 can be locked in position with respect to the delivery component 116. Forward pressure is applied to the electrosurgical device 106 and the transseptal guidewire 108 is actuated to apply the RF energy to the electrode 140 and puncture the fossa ovalis. The RF energy punctures the fossa ovalis and creates an aperture in the fossa ovalis. The transseptal guidewire 108 is unlocked from the delivery component 116, and the transseptal guidewire 108 is extended through the aperture. In general, the transseptal guidewire 108 is extended longitudinally for several millimeters prior to the distal portion 134 curving to assume a J-tip or pigtail shape and deflecting away from the atrial septum. The transseptal guidewire 108 can be advanced into the left atrium of the heart and anchored. In the embodiment of the delivery component 116 configured as the dilator/sheath assembly, the distal tapered portion 150 of a dilator, the distal tapered portion 124 is advanced into the puncture site to expand the aperture. The delivery component 116 can be retracted from the patient over the transseptal guidewire 108, and transseptal guidewire 108 can provide support for the installation of tubular members or other catheters and for advancing other devices within the heart.

An issue with typical electrosurgical transeptal guidewires used with electrosurgical generators is that there is often incomplete or vague information presented regarding the states or configuration of the transseptal guidewire in a procedural workflow prior to the application of RF energy to create a puncture. Example states of the electrosurgical transeptal guidewire can include whether the guidewire is electrically connected properly, whether the transseptal guidewire is in contact with the target tissue or merely near the target tissue, whether the guidewire is within the tip of the delivery device or deeper within the delivery device, and other examples related to relative position of the electrode or electrical coupling. Further, alerts generated during RF delivery can be vague and provide users with limited troubleshooting information.

Applicants have discovered that various states of the electrosurgical device create different electrical loads at the device connectors 104, 105 as determined by impedance measurements. Rather than apply a high-power RF puncture signal to determine the impedance measurements, the impedance measurements are based on a low-power, high frequency test signal presented to the active electrode, or to an active connector 104 on the electrosurgical generator 102 such as active connector 104. The low-power, high frequency test signal is of a lower power than the RF puncture signal. In one example, the RF puncture signal is approximately 10 Watts. In one embodiment, the low power, high frequency test signal is provided at signal levels that are high enough to perform impedance measurements with the electrosurgical generator 102, such as to make an electrical path with a current from the puncture electrode 140 through the patient to the patch electrode and return connector 105, at a frequency level high enough to avoid stimulation of cardiac tissue during the measurement (over about 100 kHz), and of a power low enough to avoid an adverse effect to the patient from the test signal. In one embodiment, the test signal is of 300 millivolts peak-to-peak at about 200 kHz. Thus, the impedance measurements can be taken at a time different than the time of the RF puncture, such as prior to application of a puncture signal.

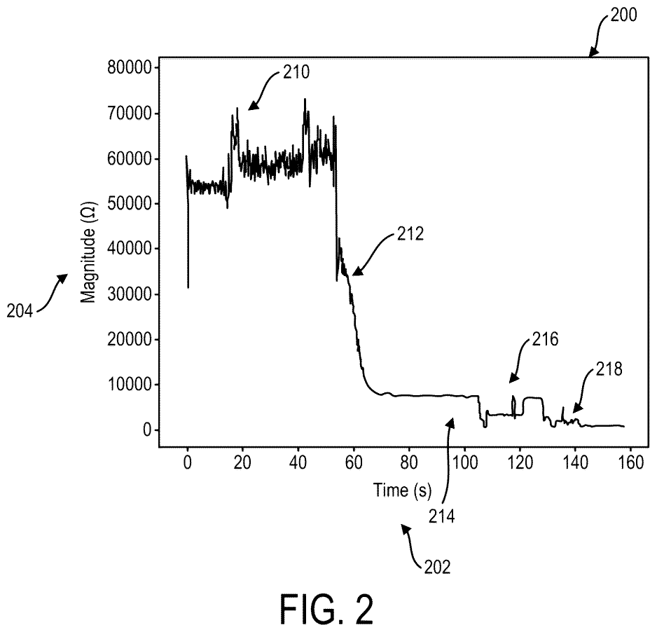

FIG. 2 illustrates a graph of impedance magnitude 200 as measured at the electrosurgical generator 102 of an example workflow of the electrosurgical transseptal guidewire 108 as a function of time as the transseptal guidewire 108 is used to reach and contact target tissue. In the example, the measured impedance magnitude is based on a low-power, high frequency test signal presented to the active connector 104 as the puncture electrode 140 of the transseptal guidewire 108 is used to reach a fossa ovalis in a heart chamber. Time of an example procedure in seconds 202 is presented along the x-axis and impedance magnitude in Ohms 204 is presented along the y-axis. At an early stage of the procedure, the transseptal guidewire 108 is electrically disconnected from electrosurgical generator 102, and a relatively high impedance magnitude is measured by the electrosurgical generator 102 at 210. As the transseptal guidewire 108 is properly electrically connected to the electrosurgical generator 102 and the puncture electrode 140 of the transseptal guidewire 108 is advanced through a dilator/sheath assembly delivery component 116, the impedance magnitude precipitously drops in value on the graph 200 at 212. The magnitude of the impedance drops again on the graph 200 as the puncture electrode 140 of the transseptal guidewire 140 initially extends from the distal tip 120 of the delivery component 116 to tent the fossa ovalis at 214. The magnitude of the impedance rises on the graph 200 as the puncture electrode 140 of the transseptal guidewire 108 retracts into the distal tip 120, such as the distal tapered portion 124 of the dilator, at 216. The magnitude of the impedance drops again on the graph 200 as the puncture electrode 140 of the transseptal guidewire 108 extends from the distal tip 120 of the dilator and returns to tent the fossa ovalis at 218.

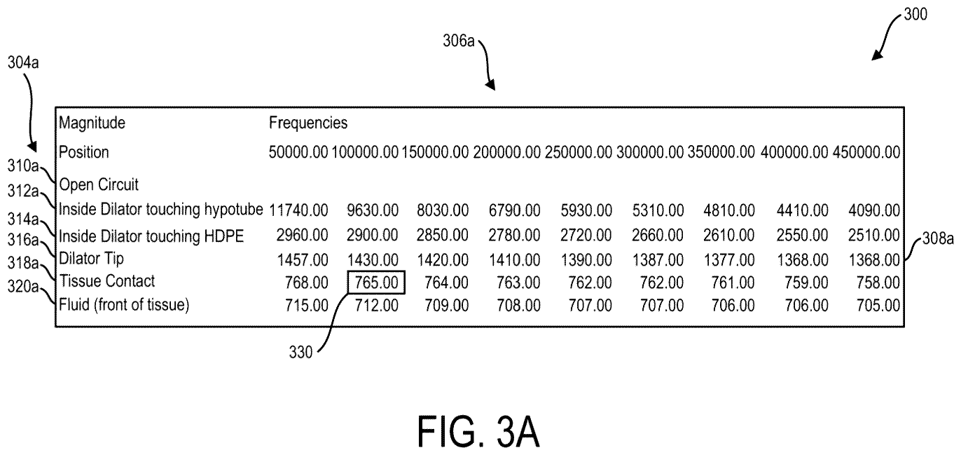

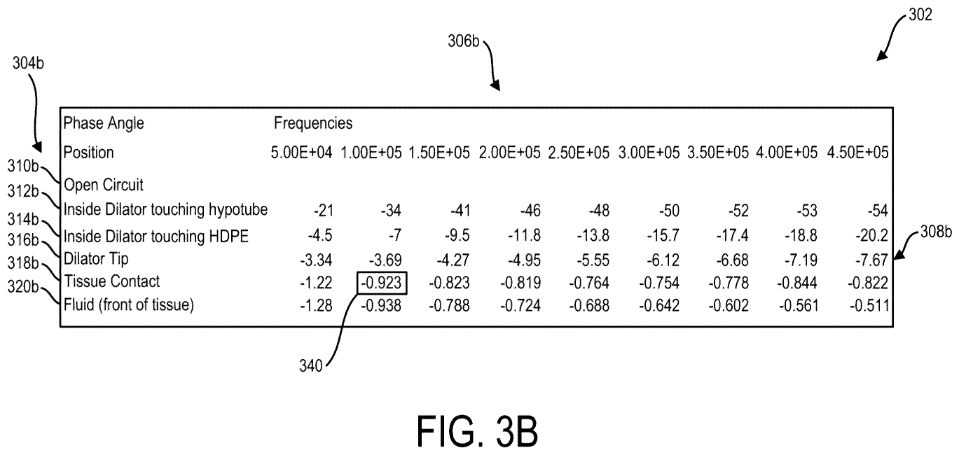

FIGS. 3A and 3B illustrate tables 300, 302, of impedance measurements based on low-power, high frequency test signals presented to the active connector 104 of the electrosurgical generator 102 associated with the electrosurgical transseptal guidewire 108 in different states, such as states within a workflow of a transseptal surgical procedure. FIG. 3A presents table 300 having measurements of complex impedance magnitude at various states in rows 304a of the transseptal guidewire for a test signal at selected frequencies in columns 306a. FIG. 3B presents table 302 having measurements of phase angle at the various states in rows 304b of the transseptal guidewire for a test signal at the selected frequencies in columns 306b. The various states in rows 304a, 304b include open circuit 310a, 310b (such as the transseptal guidewire 108 or cable 112 not properly electrically connected to the electrosurgical generator 102), puncture electrode positioned proximally in dilator/sheath assembly (puncture electrode inside dilator touching hypotube of a dilator/sheath assembly 312a, 312b), puncture electrode positioned distally in dilator/sheath assembly or near the distal tip of the dilator/sheath assembly (puncture electrode inside dilator touching the distal end portion of the dilator/sheath assembly 314a, 314b), puncture electrode at dilator tip of the dilator/sheath assembly 316a, 316b, puncture electrode extended from dilator tip of the dilator/sheath assembly and in contact with target tissue 318a, 318b, and puncture electrode extended from dilator tip of the dilator/sheath assembly and in contact with fluid 320a, 320b (such as blood in between the puncture electrode and the target tissue). The various frequencies 306 used for the low-power test signal range from 50 kHz to 450 kHz. The cell 308a at the intersection of a selected row 304a and a selected column 306a in FIG. 3A represents the impedance magnitude in Ohms for the selected state in rows 304a at the selected low-power test frequency in columns 306a. For instance, the impedance magnitude for electrode extended from the dilator tip in contact with target tissue 318a at a low-power test signal of 100 kHZ is 765 Ohms at 330. The cell 308b at the intersection of a selected row 304b and a selected column 306b in FIG. 3B represents the impedance phase angle in degrees for the selected state in rows 304b at the selected low-power test frequency in columns 306b. For instance, the impedance phase angle for electrode extended from the dilator tip in contact with target tissue 318b at a low-power test signal of 100 kHZ is −0.923 degrees at 340.

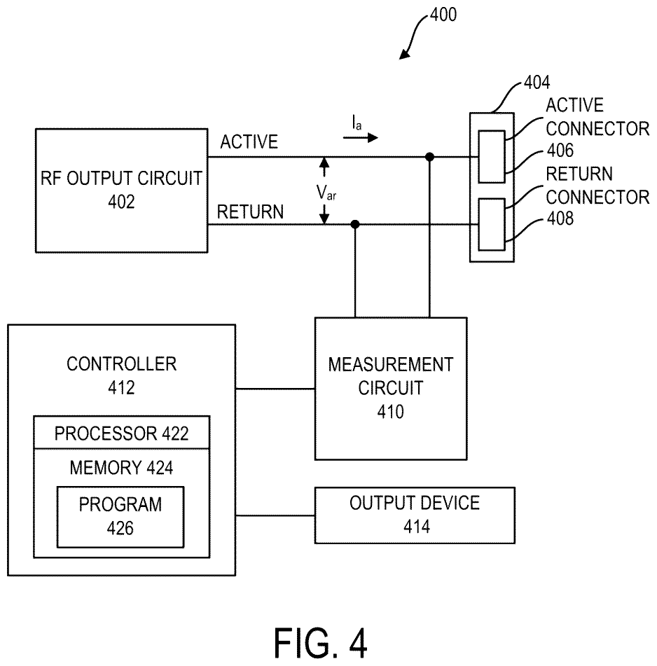

FIG. 4 illustrates an embodiment of electrosurgical unit, such as an electrosurgical generator 400, which can correspond with electrosurgical generator 102 in system 100. The electrosurgical generator 400 is configured to determine a state of an associated transseptal guide wire based on impedance measurements taken from a relatively low power, high frequency test signal. The electrosurgical generator 400 is configured to generate an RF puncture signal as well the relatively lower power RF test signal. The electrosurgical generator 400 can measure an impedance of the test signal, such as a complex impedance and phase angle, and provide detailed indications of the state of the associated electrosurgical transseptal guidewire prior to the delivery of the relatively higher power RF puncture signal. Based on the measured impedance, the electrosurgical generator 400 can determine the particular state of an associated electrosurgical transseptal guidewire, such as whether the electrosurgical transseptal guidewire is properly connected and, if so, the position of the puncture electrode with respect to a delivery component and with respect to heart tissue.

As described above by using the values populated in the tables 300, 302, from a measurement of complex impedance of a resulting low-power, high frequency test signal provided by the electrosurgical generator 400, a state of a transseptal guidewire associated with the electrosurgical generator can be determined. For example, the tables illustrate a particular state in rows 304a, 304b and a given test signal frequency 306a, 306b are used to determine an impedance measurement, such as magnitude or phase angle. Similarly, a given impedance measurement, such as magnitude or phase angle at a given test signal frequency, can be used to determine an associated transseptal guidewire state.

The electrosurgical generator 400 includes an RF output circuit 402, a plurality of device connectors 404 including an active connector 406 and a return connector 408, a measurement circuit 410, a controller 412, and output device 414. In one example, the RF output circuit 402 is configured to generate an RF puncture signal and a relatively lower power RF test signal. The RF output circuit 402 can generate an RF puncture signal and the RF test signal at the same or different frequencies. The RF output circuit 402 can include a power supply to provide a direct current signal and can convert the direct current signal to an alternating current signal. The RF output circuit 402 is configured to generate a plurality of voltages, waveforms having various duty cycles, peak voltages, crest factors, frequencies and other suitable parameters and provide the selected RF signal including the puncture signal and the test signal to the active connector 406.

The device connectors 404 can be configured to include receptacles located on a housing of the RF generator 400 that can be mechanically coupled to electrosurgical devices. The device connectors 404 are configured to electrically couple the electrosurgical generator 400 to various electrosurgical devices. For example, the active connector 406 is suitable for electrically coupling to cable 112, which can be electrically coupled to the transeptal guidewire 108. The return connector 408 is suitable for electrically coupling to the ground pad electrode 110 when an electrosurgical device is operated in a monopolar mode (or to a return electrode on the electrosurgical device when operated in a bipolar mode).

The measurement circuit 410 is electrically coupled to the device connectors 404 and is configured to determine current and voltage measurements or impedance measurements from an excitation waveform resulting from the test signal generated by the RF output circuit 402 and present the current and voltage measurements to the controller 412. The measurement circuit 410 can include circuit elements or paths electrically coupled to the RF output circuit 402 or at least some of the output connectors 404 including the active connector 406 and return connector 408 and is configured to provide a signal representative of the active and return voltages and active current. The circuit elements can include current probes to measure currents of interest. In one embodiment, the measurement circuit 410 includes an analog to digital converter coupled to the circuit elements and the controller 412 to provide digital signals to the controller 412.

The controller 412 in embodiments includes a processor 422 operably connected to a memory device 424. The memory device 424 can store processor executable instructions configured to control the processor, such as a program 426. Examples of a memory device 424 can include a non-volatile memory device such as a read only memory (ROM), electronically programmable read only memory (EPROM), flash memory, non-volatile random access memory (NRAM) or other memory device, and a volatile memory device such as random access memory (RAM) or other memory device. Memory device 224 can include various combinations of one or both of non-volatile memory devices and volatile memory devices. The processor 422 includes an output port that allows the processor 422 to control the output of or by the electrosurgical unit 400 according to a selected scheme. In some embodiments, the controller 412 includes a microprocessor or a logic processor or other control circuit such as a field programmable gate array.

Any combination of hardware and programming may be used to implement the functionalities of the electrosurgical unit 400. Such combinations of hardware and programming may be implemented in a number of different ways. For example, the programming for the electrosurgical unit 400 may be processor executable instructions stored on at least one non-transitory machine-readable storage medium, such as memory device 424 and the hardware may include at least one processing resource, such as processor 422, to execute those instructions. In some examples, the hardware may also include other electronic circuitry to at least partially implement at least one feature of electrosurgical unit 400. In some examples, the at least one machine-readable storage medium, such as a memory device 424, may store instructions that, when executed by the processor 422, at least partially implement some or all features of electrosurgical unit 400 and access data structures stored on a memory device coupled to the processor 422. In such examples, electrosurgical unit 400 may include the at least one machine-readable storage medium storing the instructions and the at least one processing resource to execute a method. The processor-executable instructions 426 may be in the form of an application, such as a computer application or module of a computer application. In other examples, the functionalities of electrosurgical unit 400 and method may be at least partially implemented in the form of electronic circuitry.

The electrosurgical generator 400 can determine the particular state of an associated electrosurgical transseptal guidewire based on the measured impedance. In one embodiment, the state of the associated electrosurgical transseptal guidewire is calculated from a formula based on the measured impedance of the associated electrosurgical transseptal guidewire. In one embodiment, the state of the associated electrosurgical transseptal guidewire is determined via a lookup table, such as a state table, stored in memory 424 and configured to be accessed by the processor 424 and program 426.

By way of illustration, using the values populated in the tables 300, 302, from a measurement of complex impedance of a resulting low-power, high frequency test signal provided by an electrosurgical generator 400, a transseptal guidewire state of a transseptal device associated with the electrosurgical generator can be determined. For example, the tables illustrate a particular state in rows 304a, 304b and a given test signal frequency 306a, 306b are used to determine an impedance measurement, such as magnitude or phase angle. Similarly, a state table can be constructed in which a given impedance measurement, such as magnitude or phase angle at a given test signal frequency can yield an associated transseptal guidewire state. Additionally, the state table or other parameters can inform the electrosurgical generator 400 as to which frequency to apply in the test signal.

In one embodiment, a state table can be constructed in which measured impedance values, such as magnitude or phase angle, for a given test signal frequency can be used to determine a given state of the transseptal guidewire. For instance, a first state table can be constructed in which a determined impedance magnitude value for a given test signal frequency can be used to determine a state from a plurality of states. A second table can be constructed in which a determined phase angle value for a given test signal frequency can be used to determine a state from a plurality of states. In one embodiment, a multidimensional table can be constructed, rather than two tables, in which inputs of impedance magnitude and phase angle can yield a signal state of the transseptal guidewire. In one embodiment, each state table can be constructed with a plurality of discrete impedance measurement values over a large range of impedance measurement values for a given test signal frequency to correspond with a transseptal guidewire state. In another embodiment, the state table can be constructed with a plurality of subranges of impedance measurement values over the large range of impedance measurement values for each given test signal frequency to correspond with the transseptal guidewire states. In one such embodiment, the subranges are distinct from each other. In another such embodiment, the subranges can include overlapping values. In this embodiment, if multiple tables are used, such as a table with overlapping ranges of impedance magnitudes and overlapping ranges of phase angles, a distinct state can be determined from a combination of measured magnitude and phase angle values.

In one example, state tables for each electrosurgical device configured for use with the electrosurgical generator 400 can be determined and stored in the memory 424 during manufacture. The controller 412 can access the state table associated with the electrosurgical device used with the electrosurgical generator 400 during a procedure. As a user changes electrosurgical devices for use with the electrosurgical generator 400, the controller 412 can access the appropriate state table memory 424. Additional or updated state tables can be loaded into memory with software and firmware updates applied to the electrosurgical generator 400, such as after a user takes possession of the electrosurgical generator 400.

In another example, the state table associated with an electrosurgical device can be stored on the memory device 109 coupled to the electrosurgical device. The controller 412 reads the memory device 109 at the time of use of the electrosurgical device to receive parameters 111 of the electrosurgical device including the state table, and loads the state table into the memory 424 as part of a device initialization at the time of attaching the electrosurgical device to the electrosurgical generator 400.

The determined state of the associated electrosurgical transseptal guidewire based on the measured impedance is provided to output device 414, which presents the state to a user. In one example, the output device 414 includes a display, and the state is presented as a visualization such as a graphic of the electrosurgical device and the puncture electrode with respect to the delivery device corresponding with the actual relative position of the puncture electrode 140 with the delivery device as determined from the measured impedance. In another example, the output device 414 is a speaker that can produce a computer-generated voice alert or other audible alert informing the user of the state.

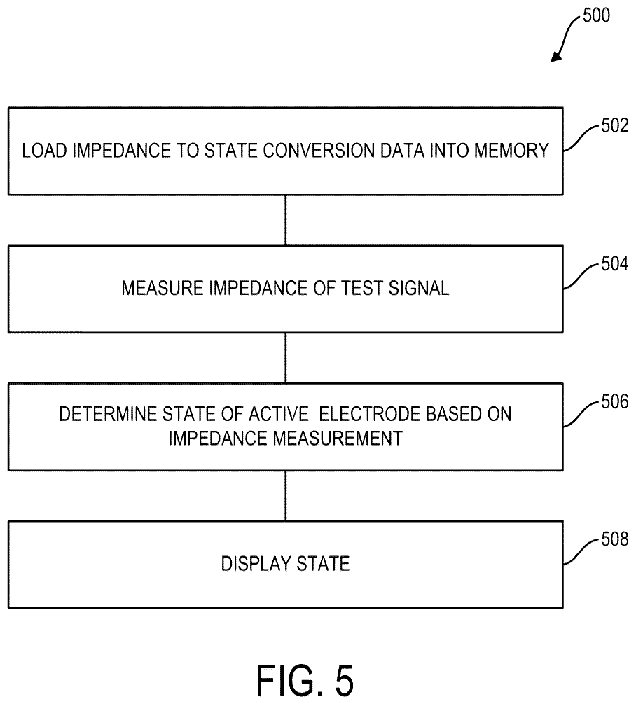

FIG. 5 illustrates a process 500 of configuring the controller 412 of the electrosurgical generator 400 to determine a state of an associated electrosurgical transseptal guidewire 108. Data regarding converting an impedance measurement to a state of the associated electrosurgical transseptal guidewire 108 is loaded into the controller 412, such as a memory 424, at 502. In one embodiment, the data may be parameter to perform a calculated conversion of the impedance measurement to a corresponding state. In another embodiment, the data may be a state table, such as a multidimensional state table. In one embodiment, the data associated with the electrosurgical transseptal guidewire is already stored in the memory 424 and loaded for use with the program upon identifying the transseptal guidewire 108 coupled to the electrosurgical generator 400. In another embodiment, the data is stored in a memory device 109 coupled to the transseptal guidewire 108. A low power, high frequency RF test signal is provided to the active connector at 502.

The impedance of the electrical load at the output connectors 404 in response to the test signal is measured at 504. The controller 412 measures an impedance of a test signal applied to the active electrode (puncture electrode 140). In one embodiment, the controller 412 can configure the RF output circuit 402 to generate and deliver the test signal to the active connector 406 at a selected frequency. The RF output circuit 402 can continuously provide the test signal and interrupt the test signal when a user selected high power, high frequency RF puncture signal is provided to the active connector 406 to puncture tissue. In another embodiment, the RF output circuit 402 can provide the test signal as a selected discrete pulse, or train of pulses, for a short period of time. In the another embodiment, the short pulse allows for other signals to be provided to the device connectors 404. The measurement circuit 410 receives the resultant test signal and, in one embodiment, converts the analog signal to a digital signal for the controller 412 to process. The measurement circuit 410 electrically coupled to the output connectors 404 can determine current waveforms and voltage waveforms of the resultant test signal, such as current at the active connector 406, Iₐ, and voltage across the active connector 406 and return connector 408, Vₐᵣ, perform an analog to digital conversion of the determination, and provide the digitized measurement to the controller 412. In one embodiment, the measurement circuit 410 determines both the voltage Vₐᵣ and current Iₐ to determine both the magnitude and phase angle for the complex impedance of the electrical load coupled to the output connectors 404. In one embodiment, the controller 412 can determine complex impedance measurements via a Fourier transform technique on the voltage Vₐᵣ and current Iₐ data of the measured test signal. In other embodiments, the complex impedance can be determined via under-sampling/over-sampling the voltage Vₐᵣ and current Iₐ to create a harmonic equivalent of the resultant waveform of the test signal and performing a sum-of-least-squares calculation to measure root-mean-square equivalents of the waveform, which can be used to calculate impedance. In still another embodiment, hardware can be applied to convert the voltage Vₐᵣ and current Iₐ to equivalent direct current signals, which is used to extract phase information. In still other embodiments, the controller 412 can apply other mechanisms and processes to determine complex impedance.

The resulting impedance measurement is applied to determine a state of the active electrode at 506. In one embodiment, the measured impedance is mapped to an associated state of the active electrode from a plurality of available states of the active electrode. For instance, impedance magnitude and phase angle are applied to a multidimensional look up table to obtain a corresponding state of the active electrode. In one embodiment, states of the active electrode include open circuit, electrode positioned within sheath, electrode positioned within dilator, electrode at dilator tip, electrode in contact with tissue, and electrode in contact with fluid. In one embodiment, the look-up table can include codes corresponding with states of the active electrode. The determined magnitude and phase angle of the complex impedance input into the look up table will yield a code that the controller converts to the associated state of the active electrode.

The state is displayed on a display device as an output at 508. Based on the determined region of interest at 506, the controller 412 generates an output corresponding with the determined state of the active electrode at 508. In one embodiment, the output can be a visualization such as string printed to a display device or a computer-generated voice output. For instance, if the determined state is an open circuit, the controller can print “Open Circuit. Check Electrical Connects to Guidewire” on a display device. If the determined state of the electrode is that the electrode is within the dilator tip, the controller can generate a graphic showing a dilator with a red light flashing within the dilator tip on the display device. The display of the state can be accompanied by another output, such as an audio output including a computer-generated spoken phrase. In one embodiment, each state of the set of state can include an associated output.

While embodiments of the disclosure are described with reference to punctures in tissue and transseptal punctures for illustration, the features of the disclosure can be used with other medical interventional procedures using RF generators.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A tissue puncture system for accessing a left atrium of the heart through a transseptal puncture, comprising:
a radiofrequency generator configured to generate radiofrequency energy;
a transseptal guidewire configured to deliver radiofrequency energy from the radiofrequency generator to tissue via an active electrode;
a controller configured to:
measure impedance during energy delivery using the active electrode, wherein the measured impedance includes complex impedance having magnitude and phase angle information;
determine the state of the active electrode from a plurality of states based on the measured impedance, wherein the state of the active electrode is based on the position of the active electrode with respect to the tissue; and
control an output of the radiofrequency generator based on the determined state of the active electrode.

2. The tissue puncture system of claim 1, wherein the active electrode is disposed in a dilator/sheath assembly.

3. The tissue puncture system of claim 2, wherein the state of the active electrode is based on a position of the active electrode with respect to the dilator/sheath assembly.

4. The tissue puncture system of claim 2, wherein the plurality of states include at least two of:
open circuit;
transseptal guidewire not properly connected;
active electrode positioned proximally in the dilator/sheath assembly;
active electrode positioned distally in the dilator/sheath assembly;
active electrode positioned at a dilator tip of the dilator/sheath assembly;
active electrode extended from the dilator tip of the dilator/sheath assembly and in contact with target tissue; or
active electrode extended from the dilator tip of dilator/sheath assembly and in contact with fluid.

5. The tissue puncture system of claim 1, wherein to determine the state, the controller is configured to compare the measured impedance values against predetermined values stored in memory, each predetermined value corresponding to one of the plurality of states.

6. The tissue puncture system of claim 1, wherein the radiofrequency generator is configured to couple to an active electrode of the transseptal guidewire and configured to couple to a return electrode.

7. The tissue puncture system of claim 6, wherein the controller includes a measurement circuit configured to obtain data from the active electrode and the return electrode.

8. The tissue puncture system of claim 7, wherein the measurement circuit is configured to monitor complex impedance including magnitude and phase angle information of the active electrode.

9. The tissue puncture system of claim 6, wherein the tissue puncture system is configured to operate in a monopolar mode.

10. The tissue puncture system of claim 1, wherein the radiofrequency generator includes one or more of a display device, user interface, connectors for the transseptal guidewire, connectors for the return electrode, speakers and lights.

11. The tissue puncture system of claim 1, wherein the radiofrequency generator provides a visual or audible indication of the determined state of the active electrode.

12. The tissue puncture system of claim 1, wherein the transseptal guidewire includes a memory device, the memory device being capable of storing parameters associated with the transseptal guidewire and being capable of being read and/or written to by the radiofrequency generator.

13. The tissue puncture system of claim 1, wherein the controller is configured to determine the state of the active electrode based on a detected change in the monitored phase angle during energy delivery.

14. The tissue puncture system of claim 1, wherein to determine the state of the active electrode from the plurality of states, the controller is configured to determine a change from a tissue contact state to a blood contact state and to control the output of the radiofrequency generator based on the detected change from the tissue contact state to the blood contact state.

15. A tissue puncture system for accessing a left atrium through a transseptal puncture, comprising:
a radiofrequency generator configured to generate radiofrequency energy;
a transseptal guidewire configured to deliver radiofrequency energy from the radiofrequency generator to tissue via an active electrode;
a measurement circuit configured to monitor complex impedance including magnitude and phase angle information of the radiofrequency energy during energy delivery;
a controller configured to:
determine a state of the active electrode from a plurality of states based on the monitored phase angle during energy delivery, wherein the plurality of states includes a delivery assembly state where the active electrode is positioned in a delivery assembly, a tissue contact state where the active electrode is in tissue contact, and a blood contact state where the active electrode is in blood contact;
determine a change in the state of the active electrode from the tissue contact state to the blood contact state; and
in response to determining the transition from the tissue contact state to the blood contact state, control an output of the tissue puncture system.

16. The tissue puncture system of claim 15, wherein the controller is configured to provide an indication of the determined state to a user using a display device.

17. The tissue puncture system of claim 15, wherein the plurality of states includes the active electrode positioned proximally in a delivery assembly, the active electrode positioned distally in the delivery assembly, the tissue contact state, and the blood contact state.

18. The tissue puncture system of claim 15, wherein the controller is configured to continuously monitor the phase angle during the transseptal puncture and to detect changes in the phase angle indicative of transitions between the plurality of states.

19. A method of controlling a tissue puncture system for accessing a left atrium of the heart through a transseptal puncture, comprising:
delivering radiofrequency energy to tissue via an active electrode of a transseptal guidewire during a transseptal puncture procedure;
measuring, using a measurement circuit, complex impedance including phase angle of electrical impedance using the active electrode during energy delivery;
determining, using a controller, a state of the active electrode from a plurality of states based on the measured phase angle, wherein the plurality of states includes a tissue contact state and a blood contact state; and
controlling, using the controller, an output of the tissue puncture system based on a determined transition from the tissue contact state to the blood contact state.

20. The method of claim 19, comprising providing a visual or audible indication of the determined state to a user using an output device.

21. The method of claim 19, comprising providing a visual or audible notification of the detected transition from the tissue contact state to the blood contact state using an output device.

22. The method of claim 19, wherein determining the state of the active electrode includes determining the state of the active electrode based on a detected change in the measured phase angle during energy delivery.

23. The method of claim 19, wherein determining the state of the active electrode includes comparing the measured complex impedance values against predetermined values stored in memory, each predetermined value corresponding to one of the plurality of states and determining the state of the active electrode based on the comparison.

24. A tissue puncture system for accessing a left atrium of the heart through a transseptal puncture, comprising:
a radiofrequency generator configured to generate radiofrequency energy;
a transseptal guidewire configured to deliver radiofrequency energy from the radiofrequency generator to tissue via an active electrode;
a measurement circuit configured to measure complex impedance including a phase angle using the active electrode during energy delivery; and
a controller configured to:
determine a state of the active electrode from a plurality of states based on the measured phase angle, wherein the plurality of states includes a tissue contact state and a blood contact state; and
generate an output corresponding to the determined state.

25. The tissue puncture system of claim 24, wherein the controller is configured to:
detect a transition from the tissue contact state to the blood contact state; and
in response to the detected transition from the tissue contact state to the blood contact state, control an output of the tissue puncture system.

26. The tissue puncture system of claim 24, wherein the controller is configured to provide a visual or audible notification of the detected transition from the tissue contact state to the blood contact state using a display device.

27. The tissue puncture system of claim 24, wherein the controller is configured to determine the state of the active electrode based on a detected change in the measured phase angle during energy delivery.
